(19) United States
(12) Patent Application Publication
(10) Pub. No.: US 2025/0144438 A1
(43) Pub. Date: May 8, 2025

(54) MERIDIAN CONDITIONING APPARATUS, CONTROL METHOD, AND METHOD FOR DETERMINING FLOW DIRECTION OF MERIDIAN QI

(71) Applicant: Wenjun FU, Beijing (CN)

(72) Inventor: Wenjun Fu, Beijing (CN)

(21) Appl. No.: 18/037,509

(22) PCT Filed: Nov. 8, 2021
(86) PCT No.: PCT/CN2021/129236

§ 371 Date: May 17, 2023

(30) Foreign Application Priority Data
Nov. 18, 2020 (CN) 202011290534.4
Nov. 8, 2021 (WO) PCT/CN2021/129236

(51) Int. Cl. A61N 2/06; A61N 2/00; A61N 2/12

(52) U.S. Cl. CPC A61N 2/06; A61N 2/002; A61N 2/004; A61N 2/12

(57) ABSTRACT

A meridian conditioning apparatus may include a control system, an information acquisition apparatus, a connection controller, a magnetic field guider, a magnetic field generator, a magnetic field introducer, and a power source. The control system comprises a central processing unit. The information acquisition apparatus comprises an input device, and an output device connected to the central processing unit. The connection controller is connected to the central processing unit, and to the magnetic field introducer and the magnetic field guider, so as to control a connection manner therebetween. The magnetic field generator is arranged around the magnetic field guider and selectively controlled by the central processing unit.

### TECHNICAL FIELD

The present disclosure relates to a meridian conditioning apparatus, and in particular to an apparatus for conditioning a meridian by introducing a magnetic field regularly at a fixed position along a fixed direction, a control method of the apparatus, and a method for determining a flow of meridian qi using the apparatus.

### BACKGROUND

According to records in ancient Chinese books, the running direction of meridian qi in the twelve meridians of a human body follows a specific law, that is, the heart meridians on two sides of the human body both run from the chest to the hands; the Sanjiao meridians both run from the hands to the head; the stomach meridians both run from the head to the feet; the liver meridians both run from the feet to the chest, and the like. However, the latest research shows that when the twelve viscera take turns to enter the master duty period (also referred to as the vigorous period of meridian qi) in a fixed order, the flow direction of meridian qi in one of two side meridians to which each viscus belongs is reversed. Moreover, a special situation occurs during the master duty period of the Sanjiao. Particularly, since May 2020, new variations of the flow direction of the meridian qi in the meridians appear. Therefore, there is an urgent need for scientific and effective methods and instruments to objectively and accurately monitor and determine the running direction of the meridian qi in the meridians of the human body, so as to determine the position of a meridian to be conditioned, the meridian qi direction to be conditioned, and conditioning time on the basis of the running law of the meridian qi.

Currently, various products for conditioning the meridians of the human body are present in the market. Some products condition the meridians by applying weak currents to the meridians and acupoints, such as biological electrotherapy. Some products condition the meridians and acupoints by physically stimulating the acupoints, such as acupoint massage or acupuncture therapy. Some products condition the acupoints by applying magnets, such as magnetic therapy pens.

Upon research, the inventor found that the running of the meridian qi in the meridians of the human body is time-ordered and directional. Therefore, only by taking external conditioning measures that conform to the running law of the meridians of the human body can good effects be achieved. As is well known, the human body is a complex electrolyte environment, and the acupoints are typically hubs for communicating a plurality of meridian stems. Therefore, the methods of applying current stimulation to the acupoints, such as the biological electrotherapy instrument, cannot achieve the purpose of promoting the ordered flow of the meridian qi. The application of the magnets to the acupoints may have a good conditioning effect on a biological field around the acupoints during a short period of time, but over time, it is prone to hinder the normal flow of the meridian qi in the meridians. The physical stimulation on the acupoints likewise has little effect on promoting the smooth running of the meridian qi in the meridians due to the lack of directional stimulation on the acupoints. Therefore, in response to the public demand for health conditioning and health care, there is an urgent need for a conditioning device capable of adjusting the meridian conditioning solution according to the running law of the meridian qi in the meridians.

### SUMMARY

In view of the defects in the prior art, the technical problem to be solved by the present disclosure is to provide an apparatus capable of conditioning meridians according to a running law of meridian qi in the meridians.

In order to achieve the above purpose, the present disclosure provides a meridian conditioning apparatus, including:
a control system comprising a central processing unit;
an information acquisition apparatus comprising an input device and an output device, wherein the output device is connected to the central processing unit;
at least one connection controller connected to the central processing unit separately;
at least one magnetic field guider composed of a wedge-shaped conductor having a gradually converging border and connected to the connection controller; and
at least one magnetic field introducer connected to the connection controller, wherein the connection controller controls a connection manner between the magnetic field introducer and the magnetic field guider or controls a connection manner between the different magnetic field introducers;
the control system receives a signal from the information acquisition apparatus through the central processing unit and controls at least the connection controller through the central processing unit, thereby controlling the connection manner between the magnetic field introducer and the magnetic field guider.

Further, the magnetic field guider is a conical spiral conductor.

Further, the magnetic field introducer is divided into a front end magnetic field introducer and a terminal end magnetic field introducer, the front end magnetic field introducer and the terminal end magnetic field introducer are connected to two ends of the magnetic field guider respectively, and a connection manner is controlled by the connection controller.

Further, the magnetic field introducer includes a wearable device and buried wires, and the buried wires are disposed in the wearable device, provided with buried points at one end, and connected to the magnetic field guider at the other end.

Further, the meridian conditioning apparatus includes a magnetic field generator disposed around the magnetic field guider.

Optionally, the magnetic field generator includes an electrical apparatus and a permanent magnet connected to the electrical apparatus, the electrical apparatus is connected to the central processing unit, and the central processing unit controls the electrical apparatus to drive the permanent magnet to move, thereby generating a varying magnetic field.

Optionally, the magnetic field generator includes an elastic conical spiral conductor that is fixed at two ends but can shake axially under an external force.

Further, the meridian conditioning apparatus also includes a sound controller or a light controller, wherein the central processing unit applies sound vibration stimulation or light radiation stimulation at a position of the magnetic field introducer by controlling the sound controller or the light controller.

In order to achieve the purpose, the present disclosure also relates to a control method of the meridian conditioning apparatus, including the following steps:
step I: acquiring, by the information acquisition apparatus, a position of a meridian to be conditioned and a running status of meridian qi in the meridian, and transmitting the position and the running status to the central processing unit;
step II: determining, by the central processing unit, a running direction of the meridian qi in the meridian;
step III: controlling, by the central processing unit according to the running direction of the meridian qi in the meridian, the connection controller to execute the following instructions:
if the running direction of the meridian qi is gradually away from the wearable device along the buried wires of the magnetic field introducer, the connection controller controls an end that is of the buried wire coinciding with the meridian and that is away from the buried point to be connected to a minimum-border end of a magnetic field guider, such that a magnetic field can be introduced along a meridian running direction; or
if the running direction of the meridian qi is gradually close to the wearable device along the buried wires of the magnetic field introducer, the connection controller controls an end that is of the buried wire coinciding with the meridian and that is away from the buried point to be connected to a maximum-border end of the magnetic field guider, such that a magnetic field can be introduced along a meridian running direction; and
step IV: sending, by the central processing unit according to the position of the meridian acquired by the information acquisition apparatus, an instruction of: controlling the connection controller to enable or disable a connection between the magnetic field guider and the buried wire in the magnetic field introducer coinciding with the meridian, thereby controlling introduction time of the magnetic field; or receiving, by the magnetic field generator, an instruction from the central processing unit to control operation time of the electrical apparatus, thereby controlling introduction time of the magnetic field.

The present disclosure also relates to a method for determining a flow direction of meridian qi with the above meridian conditioning apparatus, including the following specific steps:
step I: acquiring, by the input device, meridian qi flow signals of at least two points on a meridian on one side of the life body under test, the two points being spaced apart by a distance;
step II: obtaining, by the input device, a signal generation sequential order of the two points; and
step III: inputting, by the output device, the signal generation sequential order of the two points to the central processing unit, and determining, by the central processing unit, that the flow direction of the meridian qi is from a point generating a signal earlier to a point generating a signal later.

The present disclosure has the following advantages:
(1) The meridian conditioning apparatus in the present disclosure can introduce a varying magnetic field into the human body along the running of the meridian of the human body through the magnetic field introducer, making the position of meridian conditioning more accurate, imposing less impact on surrounding meridians, and making conditioning intensity more concentrated.
(2) The meridian conditioning apparatus in the present disclosure can adjust a direction of magnetic field introduction according to the running direction of the meridian of the human body detected by the apparatus, so as to achieve the purpose of dredging the meridian.
(3) The meridian conditioning apparatus in the present disclosure can adjust the time of magnetic field introduction according to the running direction of the meridian of the human body detected by the apparatus, so as to condition the meridian at an optimal conditioning time.
(4) To sum up, regarding the meridian conditioning apparatus in the present disclosure, a conditioning solution can be made according to a running law of the meridian in the human body detected by the apparatus, so as to control the meridian conditioning apparatus to introduce the magnetic field into the human body regularly at a fixed position along a fixed direction, thereby achieving the purpose of conditioning the meridian according to the running law of the meridian qi in the meridian.

### BRIEF DESCRIPTION OF THE DRAWINGS

FIG. 1 is a schematic diagram of the present disclosure.

FIG. 2 is a schematic diagram of a connection between devices in a specific embodiment of the present disclosure.

FIG. 3 is a top view of a magnetic field guider and a magnetic field generator in a specific embodiment of the present disclosure.

FIG. 4 is an embodiment of a magnetic field generator of the present disclosure.

FIG. 5 is another embodiment of a magnetic field generator of the present disclosure.

FIGS. 6-9 are different embodiments of a magnetic field introducer of the present disclosure.

FIG. 10 is a flowchart of a control method of the present disclosure.

FIG. 11 is a flowchart of detecting a running direction of meridian qi in a meridian using the present disclosure.

FIG. 12 is a use reference diagram of connecting the meridian conditioning apparatus of the present disclosure to a human body.

### DETAILED DESCRIPTION

The technical means employed by the present disclosure to achieve the intended invention purpose is further set forth below, in conjunction with the drawings and preferred embodiments of the present disclosure.

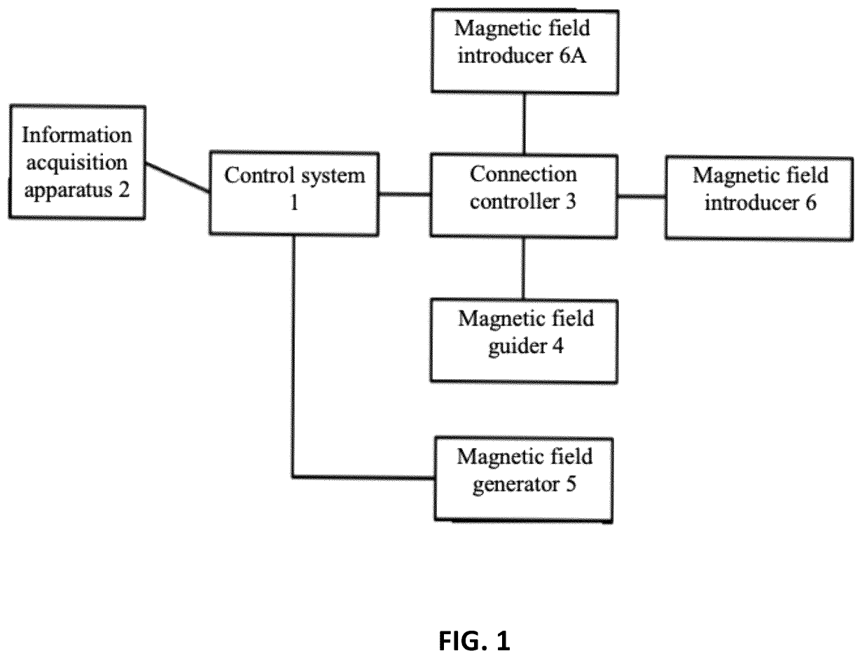

As shown in FIG. 1, a meridian conditioning apparatus involved in the present disclosure includes a control system 1, an information acquisition apparatus 2, at least one connection controller 3, at least one magnetic field generator 5, and at least one magnetic field introducer 6.

The control system 1 includes a central processing unit. The information acquisition apparatus includes an input device and an output device. The output device of the information acquisition apparatus is connected to the central processing unit and transmits acquired information to the central processing unit. Preferably, in the present disclosure, the input device in the information acquisition apparatus 2 may be configured as several sensors that can detect a signal of a flow of meridian qi in a meridian to be conditioned.

The connection controller 3 includes at least one relay. The connection controller is connected to the central processing unit and can receive an instruction from the central processing unit.

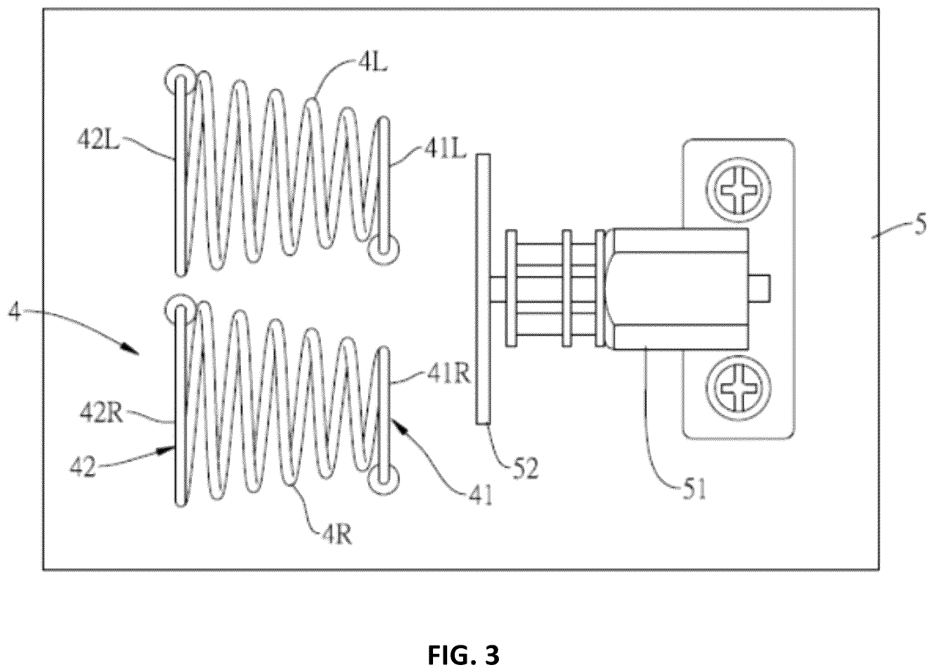

A magnetic field guider 4 is a conical spiral wire, as shown in FIG. 3. The magnetic field guider 4 includes a minimum-border end 41 and a maximum-border end 42. A magnetic field passing through the magnetic field guider 4 is directionally transmitted from the minimum-border end 41 to the maximum-border end 42. Either end of magnetic field guider 4 may be connected to the magnetic field introducer 6 through the relay in the connection controller 3. The relay in the connection controller 3 controls a connection manner between the magnetic field guider 4 and the magnetic field introducer 6.

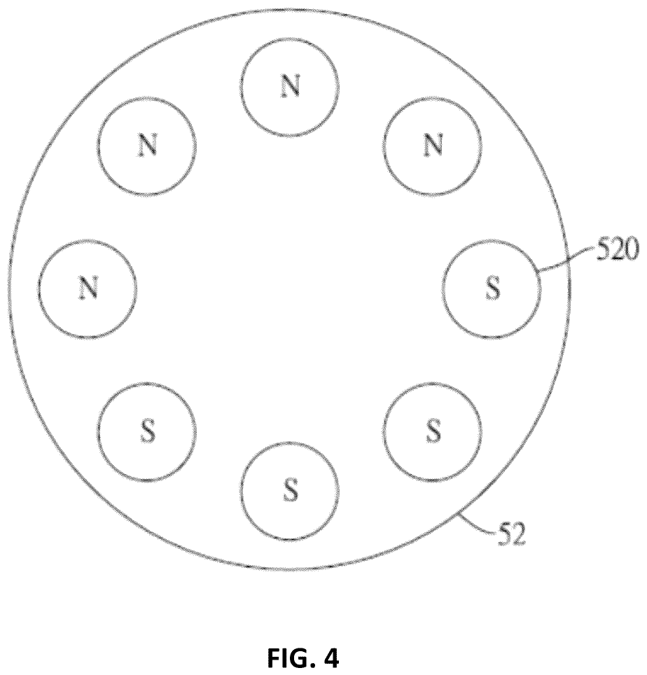

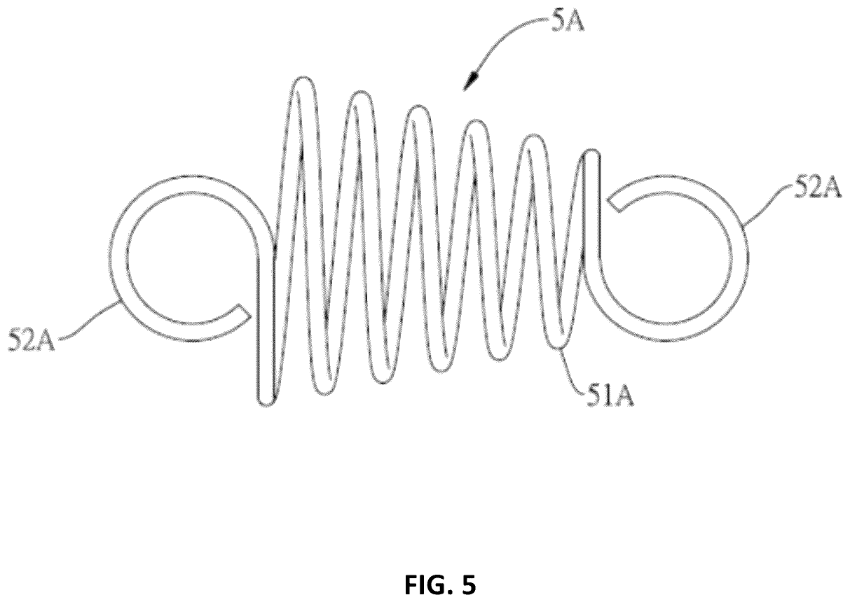

As shown in FIG. 1, the above meridian conditioning apparatus may also include the magnetic field generator 5. As shown in FIG. 3, the magnetic field generator 5 is arranged at one end of the magnetic field guider 4, and the magnetic field generator 5 includes an electrical apparatus 51 and a wheel disc 52 connected to the electrical apparatus 51. The electrical apparatus 51 may be a motor or an electrical single pendulum, as long as it can drive the wheel disc 52 to rotate or swing. As shown in FIG. 4, the electrical apparatus 51 is a motor, and a plurality of permanent magnets 520 having different magnetic polarities are disposed on the wheel disc 52. During use, the magnetic field generator 5 is connected to the central processing unit, the central processing unit controls the electrical apparatus 51 to rotate to drive the wheel disc 52 and the permanent magnets 520 disposed on the wheel disc 52 to rotate, thereby generating a varying magnetic field. FIG. 5 shows another embodiment of the magnetic field generator. In this embodiment, a body of the magnetic field generator 5A is a spring 51A made of a conductive elastic material. The spring 51A has a conical spiral shape, and provided with fixing rings 52A at two ends respectively. The magnetic field generator 5A is disposed at one end of the magnetic field guider 4 during use and is fixed by the fixing rings 52A. The spring 51A on the magnetic field generator 5A can shake with movement or vibration of the meridian conditioning apparatus, thereby inducing a varying magnetic field. If no magnetic field generator is provided, a material of at least one magnetic field guider 4 may be configured as an elastic material with two ends fixed. In this way, the at least one magnetic field guider 4 can shake with movement or vibration of the meridian conditioning apparatus, thereby inducing a varying magnetic field.

The magnetic field introducer 6 is connected to the two ends of the magnetic field guider 4 or to another magnetic field introducer 6A through the connection controller 3. Preferably, the connection controller 3 can receive the instruction from the central processing unit and control a connection position and a connection status between the magnetic field introducer 6 and the magnetic field guider 4 by changing a status of the relay therein, such that a position and a direction of magnetic field introduction can be controlled by the connection controller 3. Preferably, the magnetic field introducer 6 is divided into a front end magnetic field introducer and a terminal end magnetic field introducer, which are provided in pairs. The front end magnetic field introducer and the terminal end magnetic field introducer are connected to the two ends of the magnetic field guider 4 respectively, and the specific positions thereof are controlled by the connection controller 3. As such, the magnetic field may be simultaneously introduced to a front end and a terminal end of the meridian, achieving the purpose of simultaneously dredging the meridian at both ends along the same direction.

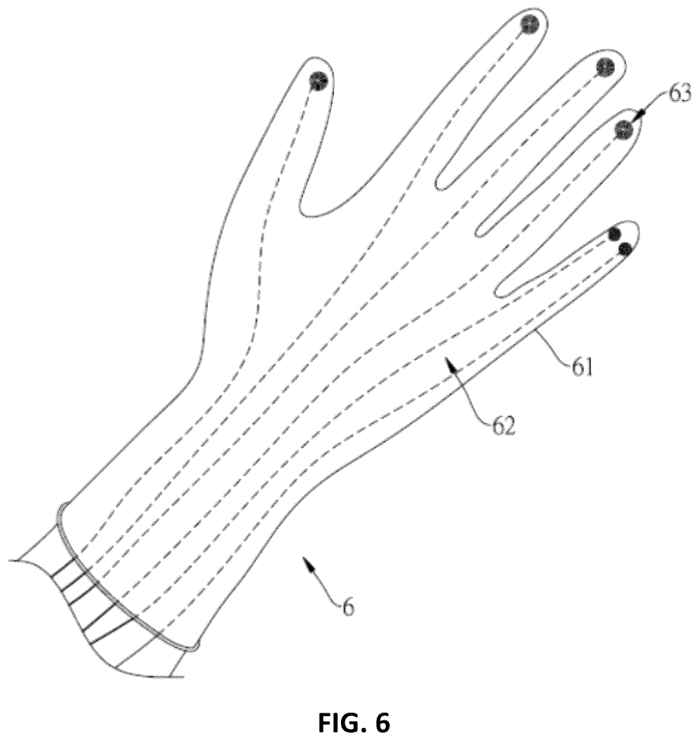

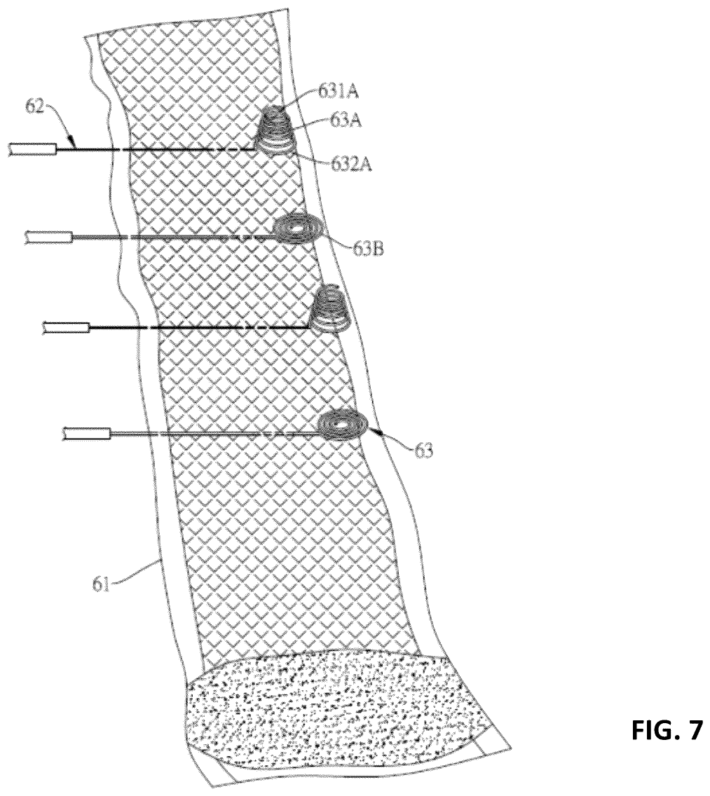

As shown in FIG. 6, the magnetic field introducer 6 includes a wearable device 61, at least one buried wire 62, and at least one buried point 63. A portion of the buried wires 62 is fixed in a lining of the wearable device 61, and the buried points 63 are disposed on the buried wires 62. At least one buried point 63 is disposed at one end of each buried wire 62, and the other ends of the buried wires 62 are connected to the minimum-border end 41 or the maximum-border end 42 of the magnetic field guider 4 through the connection controller 3. As shown in FIG. 7, the buried points 63 may include buried points 63A having stereoscopic structures and include buried points 63B having planar structures. Preferably, the buried points 63A may be conical spiral conductors, and magnetic fields passing through the buried points 63A can be transmitted from smaller-border ends 631A to larger-border ends 632A of the buried points 63A. Therefore, whether connecting the buried wires 62 to the smaller-border ends 631A or the larger-border ends 632A can be selected according to needs, so as to promote or suppress introduction of the magnetic field in a certain direction.

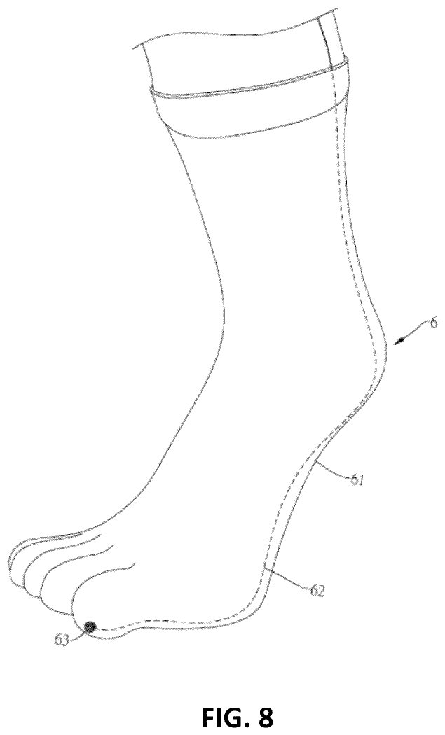

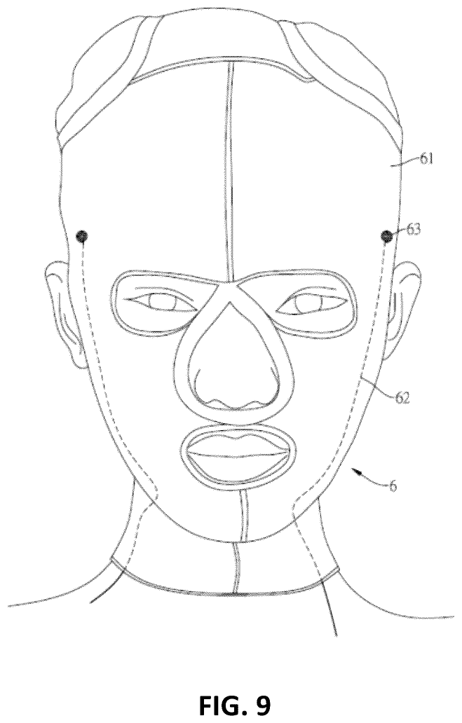

A specific form of the wearable device 61 shown in FIGS. 6-9 is not limited in the present disclosure, and may be gloves, wrist bands, socks, headgear, or underwear, as long as the buried wires 62 and buried points 63 can be fixed on a human body along a running direction of the meridian and distribution of acupoints of the human body.

Preferably, the central processing unit in the present disclosure may be further connected to a sound controller or a light controller. The central processing unit can regularly apply sound vibration stimulation or light radiation stimulation to positions of the buried wires 62 or the buried points 63 by controlling the sound controller or the light controller, thereby stimulating the acupoints or the meridian to achieve the purpose of dredging the meridian.

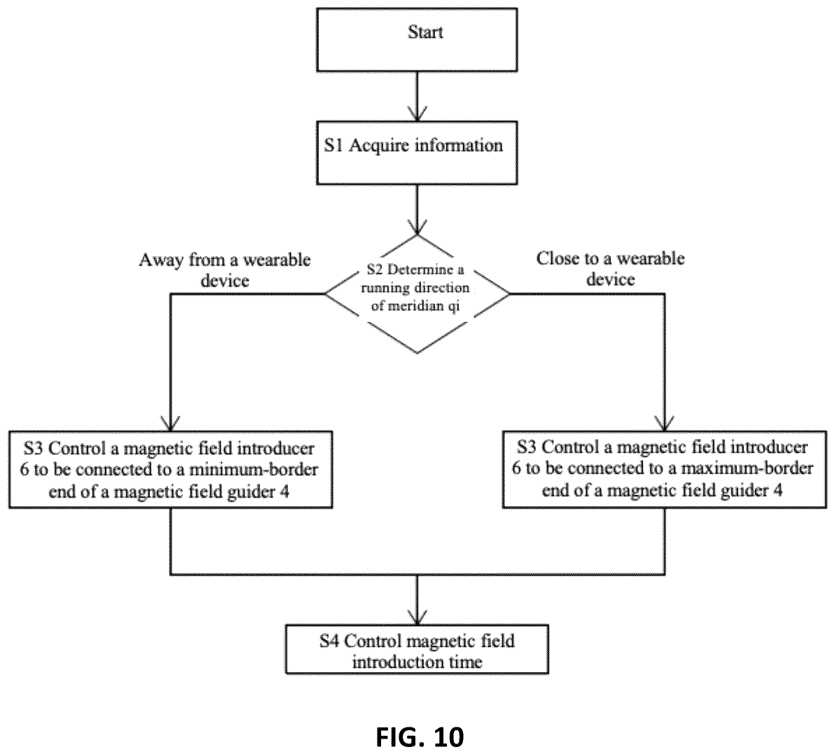

During use of the meridian conditioning apparatus in the present disclosure, a first step is to wear the wearable device 61 of the magnetic field introducer 6. After the meridian conditioning apparatus is turned on, the control system 1 controls operation of the meridian conditioning apparatus by using a control method as shown in FIG. 10, that is:
Step I: acquiring, by the information acquisition apparatus 2, a position of the meridian to be conditioned and a running status of meridian qi in the meridian, and transmitting the position and the running status to the central processing unit;
step II: determining, by the central processing unit, a running direction of the meridian qi in the meridian;
step III: controlling, by the central processing unit according to the running direction of the meridian qi in the meridian, the connection controller 3 to execute the following instructions:
if the running direction of the meridian qi is gradually away from the wearable device 61 along the buried wires 62 of the magnetic field introducer 6, the connection controller 3 controls an end that is of the buried wire 62 coinciding with the meridian and that is away from the buried point 63 to be connected to the minimum-border end 41 of the magnetic field guider 4, such that the magnetic field can be introduced along a meridian running direction; or
if the running direction of the meridian qi is gradually close to the wearable device 61 along the buried wires 62 of the magnetic field introducer 6, the connection controller 3 controls an end that is of the buried wire 62 coinciding with the meridian and that is away from the buried point 63 to be connected to the maximum-border end 42 of the magnetic field guider, such that the magnetic field can be introduced in conformity with a variation in the running direction of the meridian qi; and
step IV: sending, by the central processing unit according to the position of the meridian acquired by the information acquisition apparatus, an instruction of: controlling the connection controller to enable or disable a connection between the magnetic field guider and the buried wire in the magnetic field introducer coinciding with the meridian, thereby controlling introduction time of the magnetic field; or receiving, by the magnetic field generator, an instruction from the central processing unit to control operation time of the electrical apparatus, thereby controlling introduction time of the magnetic field.

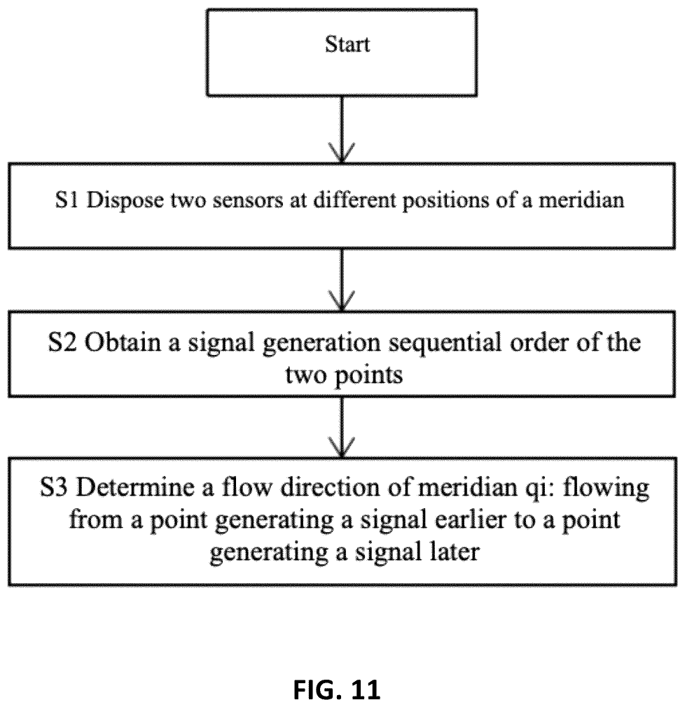

As shown in FIG. 11, a flow direction of meridian qi in a certain meridian of the human body can be determined by using the information acquisition apparatus 2. A determination method is as follows:
Step I: Several sensors are placed on a meridian on one side of the human body, where one sensor is placed at each of at least two different points, and the two points need to be spaced apart by a specific distance.
Step II: A signal generation sequential order of the two points is obtained through the at least two sensors.
Step III: The output device transmits the signal generation sequential order of the two points to the central processing unit, and the central processing unit determines that the flow direction of the meridian qi is from a point generating a signal earlier to a point generating a signal later.

### Specific Implementations:

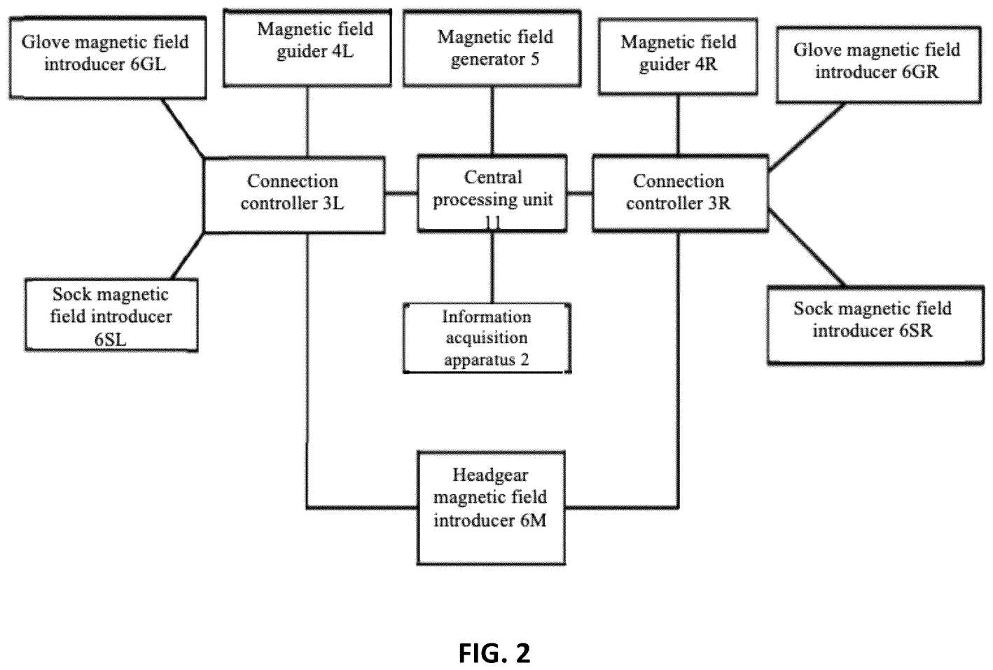

As shown in FIG. 2, the input devices provided in the information acquisition apparatus 2 in this embodiment are several sensors, and the number of the sensors may be set according to needs. The output device of the information acquisition apparatus is connected to the central processing unit 11. In this embodiment, two connection controllers, i.e., connection controllers (3L, 3R), are provided, and each of the connection controllers includes a plurality of relays therein. The connection controllers (3L, 3R) are connected to the central processing unit 11 separately.

As shown in FIGS. 2 and 3, in this embodiment, two magnetic field guiders (4L, 4R) are provided in parallel. Two ends of the magnetic field guider 4L are a minimum-border end 41L and a maximum-border end 42L respectively. Two ends of the magnetic field guider 4R are a minimum-border end 41R and a maximum-border end 42R respectively. The magnetic field generator 5 is disposed at the same end of the two magnetic field guiders (4L, 4R). The magnetic field generator 5 includes the electrical apparatus 51 and the wheel disc 52 with permanent magnets of different magnetic polarities.

In this embodiment, the magnetic field introducers are classified into glove magnetic field introducers 6G, a headgear magnetic field introducer 6M, and sock magnetic field introducers 6S according to different wearable devices. The glove magnetic field introducers 6G and the sock magnetic field introducers 6S are front end magnetic field introducers, and the headgear magnetic field introducer 6M is a terminal end magnetic field introducer. There are totally two glove magnetic field introducers (6GL, 6GR), two sock magnetic field introducers (6SL, 6SR), and one headgear magnetic field introducer (6M). As shown in FIG. 2, the connection controller 3L controls a connection manner of the magnetic field guider 4L with the headgear magnetic field introducer 6M, the glove magnetic field introducer 6GL, and the sock magnetic field introducer 6SL; and the connection controller 3R controls a connection manner of the magnetic field guider 4R with the headgear magnetic field introducer 6M, the glove magnetic field introducer 6GR, and the sock magnetic field introducer 6SR.

In this embodiment, each glove magnetic field introducer 6G includes six buried wires and several buried points; each sock magnetic field introducer 6S includes six buried wires and several buried points; and the headgear magnetic field introducer includes twenty-four buried wires corresponding to the meridians of the left and right hands and feet respectively and several buried points. During use, all the wearable devices in the magnetic field introducers (6G, 6M, 6S) are worn on the human body. The buried wires on the glove introducers (6GL, 6GR) coincide with six meridians on the left and right hands respectively, and the buried points on the glove introducers (6GL, 6GR) coincide with acupoints on the left and right hands respectively. The buried wires on the sock introducers (6SL, 6SR) coincide with six meridians on the left and right feet respectively, and the buried points on the sock introducers (6SL, 6SR) coincide with acupoints on the left and right feet respectively. Each meridian on four limbs of the human body extends to the head, and the twenty-four meridians from the limbs converge at the head. Therefore, the twenty-four buried wires on the headgear magnetic field introducer 6M coincide with the twenty-four meridians on the head respectively, and the buried points on the headgear magnetic field introducer 6M coincide with important acupoints on the head respectively. Meridian distributions on the left and right sides of the human body are symmetrical with each other. Therefore, during use of this embodiment, in order to achieve a better meridian dredging effect, the magnetic field introducers on both the left and right sides need to simultaneously introduce magnetic fields to dredge the meridians.

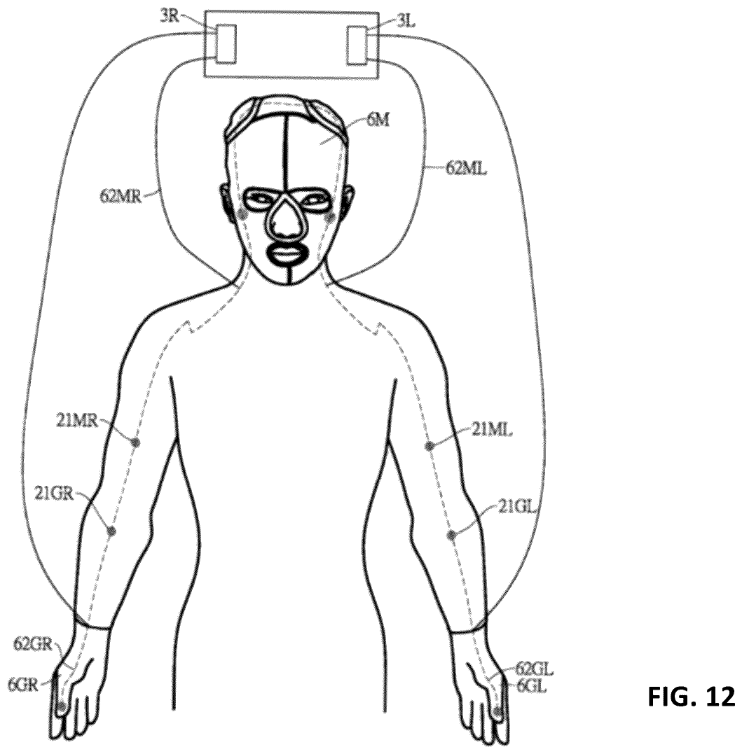

The following takes conditioning the running of the meridian qi in lung meridians as an example to introduce a working state of this embodiment during use. As shown in FIG. 12, dashed lines in the figure represent the lung meridians, the buried wire coinciding with a left-hand side lung meridian is 62GL, and the buried wire coinciding with a right-hand side lung meridian is 62GR. At the head, the buried wire coinciding with the lung meridian corresponding to the left-hand side lung meridian is 62ML, and the buried wire coinciding with a lung meridian corresponding to the right-hand side lung meridian is 62MR.

As shown in FIG. 12, first, all the magnetic field introducers need to be worn. Then four sensors in the information acquisition apparatus are placed in running directions of the lung meridians on the left and right arms separately. Two detection points are placed on the left side, one point close to the hand 21GL and the other point close to the head 21ML. Two detection points 21GR and 21MR are also placed on the other side. The sensors may also be placed within the wearable devices of the magnetic field introducer.

When the information acquisition apparatus detects that signal generation orders on both sides are from points 21ML and 21MR to points 21GL and 21GR and inputs such result to the central processing unit 11 through the output device, the central processing unit 11 determines that flow directions of the meridian qi on the left and right side arms are both from upper arms to forearms at that time.

Referring to FIG. 12 in conjunction with FIGS. 2 and 3, at that time, the central processing unit 11 sends an instruction to control the electrical apparatus 51 in the magnetic field generator 5 to rotate, thereby generating a varying magnetic field. Meanwhile, the central processing unit 11 also sends the following instructions to the connection controller 3L and the connection controller 3R.

The connection controller 3L controls the buried wire 62ML in the magnetic field introducer 6M to communicate with the minimum-border end 41L of the magnetic field guider 4L, and the connection controller 3L controls the buried wire 62GL in the magnetic field introducer 6GL to communicate with the maximum-border end 42L of the magnetic field guider 4L. The varying magnetic field generated at that time is transmitted from the minimum-border end 41L to the maximum-border end 42L of the magnetic field guider 4L and introduced to the left side lung meridian of the human body.

The connection controller 3R controls the buried wire 62MR in the magnetic field introducer 6M to communicate with the minimum-border end 41R of the magnetic field guider 4R, and the connection controller 3R controls the buried wire 62GR in the magnetic field introducer 6GR to communicate with the maximum-border end 42R of the magnetic field guider 4R. The varying magnetic field generated at that time is transmitted from the minimum-border end 41R to the maximum-border end 42R of the magnetic field guider 4R and introduced to the right side lung meridian of the human body.

Finally, the running directions of the varying magnetic fields introduced to the left and right sides of the human body are from the head to terminal ends of the left and right hands, thereby promoting a smooth flow of the meridian qi in the lung meridians along the direction from the upper arms to the forearms using the running directions of the varying magnetic fields.

Similarly, if the information acquisition apparatus acquires the flow directions of the meridian qi in the lung meridians that the flow direction of the meridian qi on the left side of the human body is still the flow direction from the upper arm to the forearm, while the flow direction of the meridian qi on the right side of the human body changes to a direction from the forearm to the upper arm, then referring to FIG. 12 in conjunction with FIGS. 2 and 3, the central processing unit 11 sends the following instructions to the connection controller 3L and the connection controller 3R:

[symbol]

The connection controller 3L controls the buried wire 62ML to be connected to the minimum-border end 41L and controls the buried wire 62GL to be connected to the maximum-border end 42L. The connection controller 3R controls the buried wire 62MR to be connected to the maximum-border end 42R and controls the buried wire 62GR to be connected to the minimum-border end 41R. The varying magnetic fields are introduced to the left side of the human body along the transmission direction from the upper arm to the forearm and to the right side of the human body along the transmission direction from the forearm to the upper arm. In this way, the magnetic fields can be introduced into the human body in conformity with a change in the running directions of the meridian qi in the meridians of the human body, thereby achieving the purpose of promoting a smooth flow of the meridian qi in the lung meridians along the flow direction.

The advantages of this embodiment are:
(1) A running law of the meridian qi in a meridian of the human body can be determined through the information acquisition apparatus 2.
(2) After the law of the meridian qi of the human body is determined, the position, direction, and time of magnetic field introduction can be adjusted according to a running direction of the meridian qi in the meridian of the human body, thereby achieving an effect of dredging the meridian according to the running law of the meridian qi of the human body.

The above descriptions are only preferred embodiments of the present disclosure, and do not impose any formal limitations on the present disclosure. Although the present disclosure has been disclosed as above with the preferred embodiments, these embodiments are not intended to limit the present disclosure. Those skilled in the art can make some changes or modifications to the above disclosed technical content into equivalent embodiments with equivalent variations, without departing from the scope of the technical solution of the present disclosure. Therefore, any simple amendments, equivalent changes, and modifications made to the above embodiments based on the technical essence of the present disclosure, without departing from the content of the technical solution of the present disclosure, still fall within the scope of the technical solution of the present disclosure.

What is claimed is:

1. A meridian conditioning apparatus, comprising:
a control system comprising a central processing unit;
an information acquisition apparatus comprising an input device and an output device, wherein the output device is connected to the central processing unit;
at least one connection controller connected to the central processing unit separately;
at least one magnetic field guider composed of a wedge-shaped conductor having a gradually converging border and connected to the connection controller; and
at least one magnetic field introducer connected to the connection controller, wherein the connection controller controls a connection manner between the magnetic field introducer and the magnetic field guider or controls a connection manner between the different magnetic field introducers;
the control system receiving a signal from the information acquisition apparatus through the central processing unit and controlling the connection controller through the central processing unit, thereby controlling the connection manner between the magnetic field introducer and the magnetic field guider.

2. The meridian conditioning apparatus of claim 1, wherein the magnetic field guider is a conical spiral conductor.

3. The meridian conditioning apparatus of claim 1, wherein the magnetic field introducer is divided into a front end magnetic field introducer and a terminal end magnetic field introducer, the front end magnetic field introducer and the terminal end magnetic field introducer are connected to two ends of the magnetic field guider respectively, and a connection manner is controlled by the connection controller.

4. The meridian conditioning apparatus of claim 1, wherein the magnetic field introducer comprises a wearable device and buried wires; and the buried wires are disposed in the wearable device, provided with buried points at one end, and connected to the magnetic field guider at the other end.

5. The meridian conditioning apparatus of claim 1, wherein the meridian conditioning apparatus comprises a magnetic field generator disposed around the magnetic field guider.

6. The meridian conditioning apparatus of claim 5, wherein the magnetic field generator comprises an electrical apparatus and a permanent magnet connected to the electrical apparatus, the electrical apparatus is connected to the central processing unit, and the central processing unit controls the electrical apparatus to drive the permanent magnet to move, thereby generating a varying magnetic field.

7. The meridian conditioning apparatus of claim 5, wherein the magnetic field generator comprises an elastic conical spiral conductor that is fixed at two ends but can shake axially under an external force.

8. The meridian conditioning apparatus of claim 1, further comprising a sound controller or a light controller, wherein the central processing unit applies sound vibration stimulation or light radiation stimulation at a position of the magnetic field introducer by controlling the sound controller or the light controller.

9. A method for controlling a meridian conditioning apparatus comprising steps of:
step I: acquiring, by the information acquisition apparatus, a position of a meridian to be conditioned and a running status of meridian qi in the meridian, and transmitting the position and the running status to the central processing unit;
step II: determining, by the central processing unit, a running direction of the meridian qi in the meridian;
step III: controlling, by the central processing unit according to the running direction of the meridian qi in the meridian, the connection controller to execute the following instructions:
if the running direction of the meridian qi is gradually away from the wearable device along the buried wires of the magnetic field introducer, the connection controller controls an end that is of the buried wire coinciding with the meridian and that is away from the buried point to be connected to a minimum-border end of a magnetic field guider, such that a magnetic field can be introduced along a meridian running direction; or
if the running direction of the meridian qi is gradually close to the wearable device along the buried wires of the magnetic field introducer, the connection controller controls an end that is of the buried wire coinciding with the meridian and that is away from the buried point to be connected to a maximum-border end of the magnetic field guider, such that a magnetic field can be introduced along a meridian running direction; and
step IV: sending, by the central processing unit according to the position of the meridian acquired by the information acquisition apparatus, an instruction of: controlling the connection controller to enable or disable a connection between the magnetic field guider and the buried wire in the magnetic field introducer coinciding with the meridian, thereby controlling introduction time of the magnetic field; or receiving, by the magnetic field generator, an instruction from the central processing unit to control operation time of the electrical apparatus, thereby controlling introduction time of the magnetic field.

10. A method for determining a flow direction of meridian qi using the meridian conditioning apparatus, wherein the meridian conditioning apparatus is connected to a life body under test, said method comprising steps of:
step I: acquiring, by the input device, meridian qi flow signals of at least two points on a meridian on one side of the life body under test, the two points being spaced apart by a distance;
step II: obtaining, by the input device, a signal generation sequential order of the two points; and
step III: inputting, by the output device, the signal generation sequential order of the two points to the central processing unit, and determining, by the central processing unit, that the flow direction of the meridian qi is from a point generating a signal earlier to a point generating a signal later.
